(19) United States
(12) Patent Application Publication
(10) Pub. No.: US 2022/0000797 A1
(43) Pub. Date: Jan. 6, 2022

(54) CLEAR PROPOFOL INJECTION AND PREPARATION METHOD THEREFOR

(71) Applicant: (BIKA BIOTECHNOLOGY (GUANGZHOU) CO., LTD), Guangzhou, Guangdong (CN)

(72) Inventor: Yingting XU, Guangzhou (CN)

(21) Appl. No.: 17/291,946

(22) PCT Filed: Nov. 7, 2018
(86) PCT No.: PCT/CN2018/114254

§ 371 Date: May 6, 2021

(51) Int. Cl. A61K 31/05; A61K 9/00; A61K 47/40; A61K 47/02

(52) U.S. Cl. CPC A61K 31/05; A61K 47/02; A61K 47/40; A61K 9/0019

(57) ABSTRACT

Disclosed are a clear propofol injection and a preparation method therefor, wherein the injection comprises the following components: propofol, cyclodextrin, stabilizer, pH regulator, and water for injection, and the pH value of the injection is 6-9, preferably 7-8.5.

### FIELD OF THE INVENTION

The invention belongs to the technical field of medicine, and specifically relates to a clear propofol injection and a preparation method therefor.

### BACKGROUND OF THE INVENTION

As a commonly used intravenous anesthetic in clinical practice, propofol has been widely used due to its fast onset, short action time, fast metabolism and high safety. Commercially available propofol emulsions comprise injection emulsions, medium and long-chain fat emulsions, and long-chain fat emulsions. Such preparations have the disadvantages of complex composition, being easy to be contaminated by bacteria, high probability of injection pain, and allergies easily caused by auxiliary materials such as soybean oil and peanut oil, which have caused serious impact on the patient's body. A propofol injection which does not use emulsions as the auxiliary material can reduce the risk of medication for patients.

There are various kinds of pharmaceutical auxiliary materials in the prior art, and there are also many insoluble drugs, but none of the auxiliary materials can solve the insoluble defects of all drugs. Fat emulsions, liposomes, lipomicrospheres, nanoparticles, albumin and the like technologies have all been used to develop propofol injections.

Since 1993, more than ten patent documents such as international patent applications PCT/GB/00737, WO/2003/06324, WO/2004/108113, WO/2007/052295, WO/2012/104730 and U.S. Pat. Nos. 7,138,387, 7,034,013, 9,006,215, etc. have disclosed the preparation of a clear propofol injection by using cyclodextrin. However, these patents have the disadvantages of high dosage of cyclodextrin, being difficult to filter, being easy to be contaminated by bacteria due to long preparation time, excessively high pH, and being difficult to store.

A clinical study in 2015 showed (Anesthesia & Analgesia 2011; 113:738-41, Crystal B. Wallentine et al.) that when sulfobutylether-β-cyclodextrin is applied to a propofol injection, the concentration of free propofol will be excessively high, and the reduction effect on the pain will be not obvious. Attempts to prepare clear propofol injections with cyclodextrin have stopped. So far, no clear propofol injection with low concentration of free propofol has been successfully developed.

### SUMMARY OF THE INVENTION

In order to prepare a clear propofol injection with a low concentration of free propofol, the applicant of the present invention screened α-cyclodextrin, β-cyclodextrin, γ-cyclodextrin and their derivatives, and finally the experimental protocols using hydroxypropyl-β-cyclodextrin and sulfobutylether-β-cyclodextrin are preferred. After attempts with a lot of auxiliary materials, we finally succeeded in preparing a clear propofol injection with a low concentration of free propofol.

One object of the invention is to provide a clear propofol injection. The injection is characterized by: being clear, low dosage of auxiliary materials, low concentration of free propofol, good stability, and being in-line filterable.

Another object of the invention is to provide a method for preparing the clear propofol injection.

The above-mentioned objects of the invention are realized by the following technical solutions.

In one aspect, the invention provides a clear propofol injection, wherein the injection comprises the following components: propofol, a cyclodextrin, a stabilizer, a pH regulator and water for injection, and the pH value of the injection is 6-9, preferably 7-8.5.

Preferably, the cyclodextrin is selected from one or more of hydroxypropyl-β-cyclodextrin, sulfobutylether-β-cyclodextrin, hydroxypropyl-sulfobutylether-β-cyclodextrin, mannosyl-β-cyclodextrin and galactosyl-β-cyclodextrin, and preferably hydroxypropyl-β-cyclodextrin or sulfobutylether-β-cyclodextrin.

Preferably, the stabilizer is selected from pharmaceutically acceptable bases or acids, including but not limited to one or more of sodium bicarbonate, potassium bicarbonate, sodium hydroxide, sodium lactate, sodium hydrogen phosphate, sodium citrate, sodium acetate, ammonium acetate, ammonium bicarbonate, potassium bicarbonate, phosphoric acid, lactic acid, citric acid, acetic acid, hydrochloric acid and carbon dioxide, and preferably sodium bicarbonate.

Preferably, the pH regulator is selected from pharmaceutically acceptable bases or acids, including but not limited to sodium bicarbonate, sodium hydroxide, hydrochloric acid and carbon dioxide.

Preferably, the amount of propofol in the injection is 0.5%-2% (w/v), preferably 1% (w/v) by weight percentage.

Preferably, the amount of the cyclodextrin in the injection is 10%-30% (w/v), preferably 12%-25% (w/v) by weight percentage.

Preferably, the amount of the stabilizer in the injection is 0.001-1.0% (w/v), preferably 0.01-1.0% (w/v), more preferably 0.01-0.5% (w/v) by weight percentage.

In the injection, the amount of the pH regulator may be tailored such that the injection can be adjusted to an appropriate pH value.

Preferably, the content of free propofol in the injection is not higher than 50 μg/mL, preferably not higher than 35 μg/mL, and most preferably not higher than 30 μg/mL.

Preferably, the injection further comprises one or more of preservatives, antioxidants, bacteriostatic agents and osmotic adjusters. These preservatives, antioxidants, and bacteriostatic agents, such as EDTA, sodium bisulfite, vitamin C, cysteine and the like common reagents, can be tailored by the persons who formulate the formulation according to process requirements.

In a preferred embodiment, the invention provides a propofol injection, wherein the injection comprises propofol, sulfobutylether-β-cyclodextrin, sodium bicarbonate, and a pH regulator other than sodium bicarbonate and water for injection, and the pH value of the injection is 6-9, preferably 7-8.5;

Preferably, the amount of propofol in the injection is 1% (w/v) by weight percentage;

Preferably, the amount of sulfobutylether-β-cyclodextrin in the injection is 12%-25% (w/v) by weight percentage.

Preferably, the amount of sodium bicarbonate in the injection is 0.01-1.0% (w/v) by weight percentage.

Preferably, in the injection, the pH regulator other than sodium bicarbonate is sodium hydroxide, hydrochloric acid, or carbon dioxide.

Compared with the existing cyclodextrin preparations, the propofol injection of the invention has a lower concentration of free propofol. Specifically, the documents of CyDex show that the concentration of free propofol in the composition of propofol and cyclodextrin is about 50-80 μg/mL, while in the invention, the concentration of free propofol in the water phase is about 30 μg/mL.

In the invention, sodium bicarbonate is used as a stabilizer, and the functions of the stabilizer include: (1) keeping the preparation clear, transparent and free of oil droplets during storage when the dosage of cyclodextrin is low; (2) reducing the concentration of free propofol in the water phase.

The invention is also characterized by reducing the amount of sodium bicarbonate as much as possible, and controlling the pH value and the amount of sodium bicarbonate within a safe range suitable for large-volume infusion.

The invention achieves the effects of pain reduction and injection safety by using a low amount of sodium bicarbonate (about 0.5 mg/mL).

The propofol injection of the invention is convenient to store and has good stability. When stored at 25° C. for 6 months, precipitation of the main drug and formation of oil droplets are not observed, and the content of the main drug propofol is not less than 99.5%.

In another aspect, the invention provides a preparation method of the above-mentioned propofol injection, the preparation method comprising the steps of: adding a prescription amount of cyclodextrin (preferably sulfobutylether-β-cyclodextrin) and a stabilizer (preferably, sodium bicarbonate) to water for injection, introducing inert gas after dissolution, adding propofol, stirring, adjusting the pH value with a pH regulator, filtering, and packaging under the protection of inert gas.

Preferably, the inert gas is nitrogen, which is used to reduce the oxygen content.

In the invention, cyclodextrin such as sulfobutylether-β-cyclodextrin is used to encapsulate the insoluble propofol. When the content of sulfobutylether-β-cyclodextrin exceeds 22 g, the concentration of free propofol no longer increases as the increase of the amount of cyclodextrin, but decreases significantly. The addition of sodium bicarbonate reduces the concentration of free propofol and reduces the amount of cyclodextrin used.

The propofol injection of the invention adopts the following methods to improve stability:

1) Propofol is loaded with cyclodextrin, such as sulfobutylether-β-cyclodextrin, to reduce the contact of propofol with oxidants;

2) Formulating and packaging are performed under a sterile nitrogen atmosphere;

3) Since propofol is more likely to be oxidized in a strong alkaline environment, sodium bicarbonate is used as a stabilizer to reduce the risk of quality degradation caused by excessive local alkalinity during the formulating process;

4) The invention also uses a pH regulator to control the pH to 7-8.5 to reduce the damage to acid-base balance in a patient caused by a large infusion of a high-pH solution.

In a preferred technical solution, the prescription of the propofol injection of the invention is as follows:

| | | |
|---|---|---|
| propofol | 1.000 | g |
| sulfobutylether-β-cyclodextrin | 16 | g |
| sodium bicarbonate | 0.001-0.5 | g |

| | |
|---|---|
| pH regulator | q.s. |
| water for injection | to 100 mL |

Preparation Method:

At room temperature, to 16 g of sulfobutylether-β-cyclodextrin and 0.001-0.5 g of sodium bicarbonate, 50 mL of water for injection was added. After dissolution by stirring, nitrogen was introduced into the solution to remove dissolved oxygen, and 1000 mg of propofol was added under a nitrogen atmosphere. After stirring for 2-6 hours, the pH value was adjusted to 6-9, and water was added to 100 mL. The resulting solution was sterilized by 0.22 μm filtration, filled into a transparent sterile ampoule, packaged under sterile nitrogen which has driven away the air, and stored at 25° C.

In a more preferred technical solution, the prescription of the propofol injection of the invention is as follows:

| | | |
|---|---|---|
| propofol | 1.000 | g |
| sulfobutylether-β-cyclodextrin | 16.0 | g |
| sodium bicarbonate | 0.05 | g |

| | |
|---|---|
| pH regulator | q.s. |
| water for injection | to 100 mL |

### Preparation Method:

At room temperature, to 16.0 g of sulfobutylether-β-cyclodextrin and 0.05 g of sodium bicarbonate, 50 mL of water for injection was added. After dissolution by stirring, nitrogen was introduced into the solution to remove dissolved oxygen, and 1000 mg of propofol was added under a nitrogen atmosphere. After stirring for 2-6 hours, the pH value was adjusted to 6-9, and water was added to 100 mL. After stirring evenly, the resulting solution was sterilized by 0.22 μm filtration, filled into a transparent sterile ampoule, packaged under sterile nitrogen which has driven away the air, and stored at 25° C.

In another aspect, the invention provides a method of anesthesia, which comprises administering the aforementioned propofol injection to a patient in need thereof.

In the invention, nitrogen is injected into the cyclodextrin solution to drive away the dissolved oxygen in the solvent system; a stabilizer is added to minimize the free propofol in the water phase, reduce the probability that the main drug precipitates and forms oil droplets during long-term storage; improve the encapsulation rate of cyclodextrin to further encapsulate propofol, thereby reducing the concentration of free propofol.

Compared with the prior art, the present application has at least the following advantageous technical effects:

The invention prepares a formulation with a combination of cyclodextrin, propofol, and sodium bicarbonate, which significantly reduces the concentration of free propofol, reduces the risk of injection pain of the injection which is a combination of propofol and cyclodextrin, and has a predictable effect on mitigating the frequency and intensity of injection pain.

Among the existing cyclodextrin patents, the present patented product has the advantages of low dosage of auxiliary materials, low nephrotoxicity, and low hemolysis.

The concentration of sodium bicarbonate in the invention is about 0.5 mg/mL, which significantly reduces the possibility of inducing severe alkalosis, hypokalemia, and hypocalcemia in normal people, and greatly improves safety.

The injection of the invention is clear, which is convenient for visual inspection before administration. An in-line microbial filter can be used for drug delivery.

The invention avoids the use of auxiliary materials such as soybean oil and lecithin which require complex processes and have a high risk of allergy, expands the applicable population of propofol injection, and reduces the risk of allergization by auxiliary materials of the propofol injection.

The injection of the invention does not contain lipids that contribute to the growth of microorganisms, and can also reduce the risk of microbial infections during long-term administration.

The injection of the invention does not contain phospholipids. Therefore, the plasma concentration of the phospholipid is not affected by the parenteral administration of the composition, and the injection can also be used by patients allergic to peanuts, soybeans and the like.

The injection of the invention will not cause any change in the elimination of triglycerides, and thus will not cause disorder of lipid metabolism.

### DESCRIPTION OF THE DRAWINGS

FIG. 1 is an enlarged view of the hydrogen nuclear magnetic resonance spectrum of the sample prepared according to Example 2;

FIG. 2 is an enlarged view of the hydrogen nuclear magnetic resonance spectrum of the sample prepared according to Example 9.

### DETAILED DESCRIPTION OF THE INVENTION

In order to further illustrate the invention, a further detailed description of the invention is provided hereinafter by way of preparation examples of a novel propofol injection with good stability, convenient storage and low injection pain.

The method for detecting propofol content in the preparation and the method for detecting the concentration of free propofol as used in the following examples are illustrated as follows:

Detection method of propofol content in the preparation:

An appropriate amount of propofol injection was precisely drawn into a volumetric flask, and chromatographscally pure methanol was add up to the mark, mixed evenly, and centrifuged. The supernatant was obtained and the total concentration of propofol in the preparation was determined by HPLC according to the chromatographic conditions under the content determination item;

Chromatographic conditions: chromatographic column: C18 4.6×250 mm, 5 μm; mobile phase:melhanol-water (80:20); detection wavelength: 280 nm; flow rate: 1 mL/min; column temperature: 25° C.

Detection method of concentration of free propofol:

The prescription amount of water for injection was replaced with heavy water to prepare the injection.

Conditions of hydrogen nuclear magnetic resonance spectrum: zg30 pulse sequence was used to obtain 1H-NMR spectra at a constant temperature (25° C). Spectral width (SWH): 8000 Hz, RF center frequency (OIP): Hz, number of sampling points (TD): 32K, sampling time (AQ): 4.09 s, relaxation time (D1): 20 s, number of samplings (NS): 64.

The calculated content is a relative percentage content of the sample and the impurity calculated according to the following equation by comparing the absorption peak area (A1/n1) caused by a proton at the para position of the phenolic hydroxyl group and the absorption peak area (A2/n2) caused by the proton at the same position on the free propofol group: the relative percentage content of the sample={(A1/n1)/[(A1/n1)+(A2/n2)]}×100% where n1, n2 are the number of the protons on the specified group.

### EXAMPLES 1-8

At room temperature, to a prescription amount of sulfobutylether-β-cyclodextrin and a corresponding prescription amount of sodium bicarbonate, 50 mL of water for injection was added. After dissolution by stirring, nitrogen was introduced into the solution to remove dissolved oxygen, and 1000 mg of propofol was added under a nitrogen atmosphere. After stirring for 2-6 hours, the pH value was adjusted to 6-9, and water was added to 100 mL. After stirring evenly, the resulting solution was sterilized by 0.22 μm filtration, filled into a transparent sterile ampoule, packaged under sterile nitrogen which has driven away the air, and stored at 25° C. The amounts of the raw materials used in each example, the appearance of the obtained injection and the concentration of free propofol are shown in Table 1.

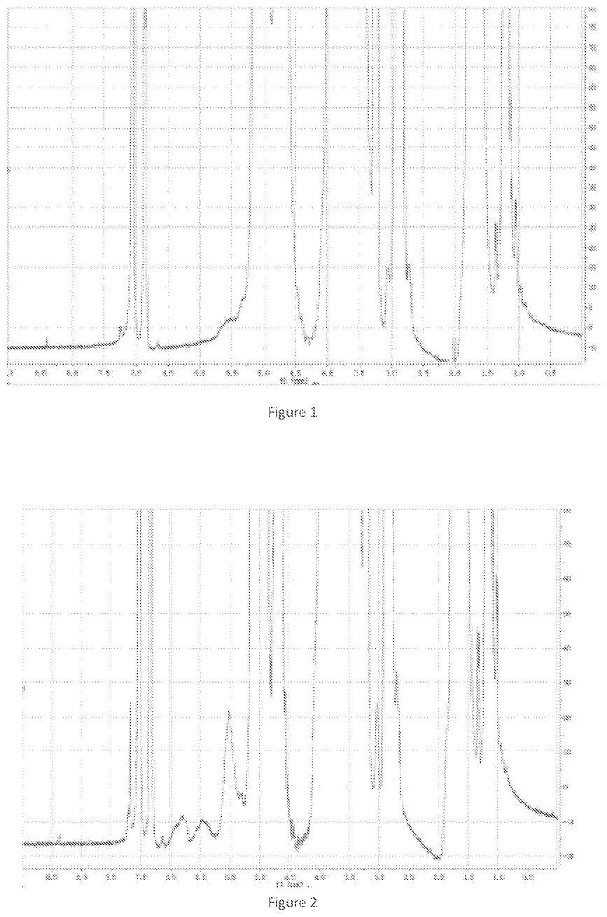

FIG. 1 shows an enlarged view of the hydrogen nuclear magnetic resonance spectrum of the sample prepared according to Example 2;

### EXAMPLES 9-10

Examples 9-10 were prepared with reference to the patents of Cydex, and specifically to Examples 1 and 2 of U.S. Pat. No. 7,034,013. FIG. 2 shows an enlarged view of the hydrogen nuclear magnetic resonance spectrum of the sample prepared according to Example 9.

The amounts of the raw materials used in each example, the appearance of the obtained injection and the concentration of free propofol are shown in Table 1.

TABLE 1

| | | | sodium | | | | free |
|---|---|---|---|---|---|---|---|
| Example | Propofol | cyclodextrin | bicarbonate | | water for | | propofol |
| No. | (g) | (g) | (g) | pH | injection | appearance | (μg/mL) |
| 1 | 1.000 | 16 | 0.01 | 7.2 | supplement | clear | 35.3 |
| 2 | 1.000 | 16 | 0.05 | 7.6 | to 100 mL | clear | 30.1 |
| 3 | 1.000 | 16 | 0.1 | 8.2 | | clear | 29.6 |
| 4 | 1.000 | 16 | 0.5 | 7.6 | | clear | 25.7 |
| 5 | 1.000 | 16 | 1.0 | 7.6 | | clear | 24.5 |
| 6 | 1.000 | 16 | 1.2 | 7.6 | | yellowish | — |
| 7 | 1.000 | 16 | 0.001 | 7.6 | | opacified | — |
| 8 | 1.000 | 16 | — | — | | opacified | — |
| 9 | 1.000 | 22 | — | — | | clear | 55.4 |
| 10 | 1.000 | 30 | — | — | | clear | 54.7 |

It can be seen from Examples 1-10 that when sodium bicarbonate was higher than 1% (w/v), the preparation is slightly yellow and the stability was poor, and when sodium bicarbonate was lower than 0.01% (w/v), the preparation was opacified. By comparing Example 9 and Example 10, it can be seen that the increase of the concentration of sulfobutylether-β-cyclodextrin can not significantly reduce the concentration of free propofol.

In Examples 8-10 without sodium bicarbonate, when sulfobutylether-β-cyclodextrin was 16 g, there were oil droplets floating on the liquid surface after standing, indicating that cyclodextrin did not effectively encapsulate propofol, and when sulfobutylether-β-cyclodextrin was 22 g, the solution was clear, indicating that at least 22 g of sulfobutylether-β-cyclodextrin was required to effectively encapsulate propofol. When sulfobutylether-β-cyclodextrin was 30 g, the solution was clear, while the concentration of free propofol did not decrease significantly. After comparison, in a prescription that uses an appropriate amount of sodium bicarbonate, the content of free propofol was significantly reduced, and the amounts of sulfobutylether-β-cyclodextrin and sodium bicarbonate used were also less.

### EXAMPLE 11

At room temperature, to 18.0 g of sulfobutylether-β-cyclodextrin and 100 mg of sodium citrate, 50 mL of water for injection was added. After dissolution by stirring, nitrogen was introduced into the solution to remove dissolved oxygen, and 1000 mg of propofol was added under a nitrogen atmosphere. After stirring for 2-6 hours, the pH value was adjusted to 6-9, and water was added to 100 mL. After stirring evenly, the resulting solution was sterilized by 0.22 μm filtration, filled into a transparent sterile ampoule, packaged under sterile nitrogen which has driven away the air, and stored at 25° C. The concentration of free propofol was measured as 37.3 μg/mL.

### EXAMPLE 12

At room temperature, to 18.0 g of sulfobutylether-β-cyclodextrin and 100 mg of meglumine, 50 mL of water for injection was added. After dissolution by stirring, nitrogen was introduced into the solution to remove dissolved oxygen, and 1000 mg of propofol was added under a nitrogen atmosphere. After stirring for 2-6 hours, the pH value was adjusted to 6-9, and water was added to 100 mL. After stirring evenly, the resulting solution was sterilized by 0.22 μm filtration, filled into a transparent sterile ampoule, packaged under sterile nitrogen which has driven away the air, and stored at 25° C. The concentration of free propofol was measured as 36.6 μg/mL.

### EXAMPLE 13

At room temperature, to 18.0 g of sulfobutylether-β-cyclodextrin and 100 mg of ammonium bicarbonate, 50 mL of water for injection was added. After dissolution by stirring, nitrogen was introduced into the solution to remove dissolved oxygen, and 1000 mg of propofol was added under a nitrogen atmosphere. After stirring for 2-6 hours, the pH value was adjusted to 6-9, and water was added to 100 mL. After stirring evenly, the resulting solution was sterilized by 0.22 μm filtration, filled into a transparent sterile ampoule, packaged under sterile nitrogen which has driven away the air, and stored at 25° C. The concentration of free propofol was measured as 35.2 μg/mL.

### EXAMPLE 14

At room temperature, to 20.0 g of sulfobutylether-β-cyclodextrin and 100 mg of ammonium acetate, 50 mL of water for injection was added. After dissolution by stirring, nitrogen was introduced into the solution to remove dissolved oxygen, and 1000 mg of propofol was added under a nitrogen atmosphere. After stirring for 2-6 hours, the pH value was adjusted to 6-9, and water was added to 100 mL. After stirring evenly, the resulting solution was sterilized by 0.22 μm filtration, filled into a transparent sterile ampoule, packaged under sterile nitrogen which has driven away the air, and stored at 25° C. The concentration of free propofol was measured as 38.7 μg/mL.

### EXAMPLE 15

At room temperature, to 10.0 g of sulfobutylether-β-cyclodextrin and 50 mg of sodium hydrogen phosphate, 50 mL of water for injection was added. After dissolution by stirring, nitrogen was introduced into the solution to remove dissolved oxygen, and 500 mg of propofol was added under a nitrogen atmosphere. After stirring for 2-6 hours, the pH value was adjusted to 6-9, and water was added to 100 mL. After stirring evenly, the resulting solution was sterilized by 0.22 μm filtration, filled into a transparent sterile ampoule, packaged under sterile nitrogen which has driven away the air, and stored at 25° C. The concentration of free propofol was measured as 33.7 μg/mL.

### EXAMPLE 16

At room temperature, to 20.0 g of sulfobutylether-β-cyclodextrin and 100 mg of sodium lactate, 50 mL of water for injection was added. After dissolution by stirring, nitrogen was introduced into the solution to remove dissolved oxygen, and 1000 mg of propofol was added under a nitrogen atmosphere. After stirring for 2-6 hours, the pH value was adjusted to 6-9, and water was added to 100 mL. After stirring evenly, the resulting solution was sterilized by 0.22 μm filtration, filled into a transparent sterile ampoule, packaged under sterile nitrogen which has driven away the air, and stored at 25° C. The concentration of free propofol was measured as 36.1 μg/mL.

### COMPARATIVE EXAMPLE 1

To 19 mL of Diprivan which is a commercially available propofol injection, 1 mL of 5% sodium bicarbonate was added. The resulting solution was filled into a transparent sterile ampoule, packaged under sterile nitrogen which has driven away the air, and stored at 25° C. The final concentration of sodium bicarbonate was calculated as 0.25%.

### COMPARATIVE EXAMPLE 2

To 15 mL of Diprivan which is a commercially available propofol injection, 5 mL of 5% sodium bicarbonate injection was added. The resulting solution was filled into a transparent sterile ampoule, packaged under sterile nitrogen which has driven away the air, and stored at 25° C. The final concentration of sodium bicarbonate was calculated as 1.25%.

### EXAMPLE 17

### Investigation of Stability of Propofol Injection

Samples were prepared according to the prescriptions of Examples 1-3 and Comparative Examples 1-2 for investigation. The samples were allowed to stand at 40° C. under a light tight environment, and were detected at different time points. The stability-related data are shown in Table 2. It can be seen from Table 2 that the propofol injection of the invention exhibits a good stability.

TABLE 2

| Time (day) | Sample | morphology | pH | Content (%) |
|---|---|---|---|---|
| 0 | Example 1 | a colorless clear | 7.2 | 100.1 |
| | | liquid | | |
| | Example 2 | a colorless clear | 7.6 | 99.9 |
| | | liquid | | |
| | Example 3 | a colorless clear | 8.2 | 99.9 |
| | | liquid | | |
| | Comparative | a white emulsion | 7.5 | 99.9 |
| | example 1 | | | |
| | Comparative | a white emulsion | 8.8 | 99.9 |
| | example 2 | | | |
| 5 | Example 1 | a colorless clear | 7.2 | 99.9 |
| | | liquid | | |
| | Example 2 | a colorless clear | 7.6 | 99.9 |
| | | liquid | | |
| | Example 3 | a colorless clear | 8.2 | 99.9 |
| | | liquid | | |
| | Comparative | a white emulsion | 7.5 | 99.9 |
| | example 1 | | | |
| | Comparative | a white emulsion | 8.8 | 98.6 |
| | example 2 | | | |
| 10 | Example 1 | a colorless clear | 7.2 | 100.0 |
| | | liquid | | |
| | Example 2 | a colorless clear | 7.6 | 99.7 |
| | | liquid | | |
| | Example 3 | a colorless clear | 8.1 | 99.5 |
| | | liquid | | |
| | Comparative | a white emulsion | 7.5 | 99.2 |
| | example 1 | | | |
| | Comparative | a yellowish | 8.8 | 95.3 |
| | example 2 | emulsion | | |

In order to further investigate the stability, samples were prepared according to the prescription of Example 2 for investigation. The samples were allowed to stand at 25° C. under a light tight environment, and were detected at different time points. The stability-related data are shown in Table 3.

TABLE 3

| | Day 0 | Day 10 | Day 30 | Day 60 | Day 180 |
|---|---|---|---|---|---|
| Appearance | colorless | colorless | colorless | colorless | colorless |
| | and clear | and clear | and clear | and clear | and clear |
| pH | 7.6 | 7.6 | 7.6 | 7.6 | 7.5 |
| Content of | 100.2% | 99.8% | 99.5% | 99.6% | 99.5% |
| propofol | | | | | |

According to the instruction of sodium bicarbonate injection, the administration concentration for clinical treatment of metabolic acidosis is 1.4% to 8%. U.S. Pat. No. 8,546,453 shows that sodium bicarbonate can reduce injection pain only when its final concentration approaches 1.4%, while a low concentration of sodium bicarbonate (0.25%) cannot reduce injection pain. However, long-term infusion of such an emulsion will greatly increase the concentration of bicarbonate in a human body, which is very likely to damage the patient's acid-base balance and cause discomfort in the patient. The invention greatly reduces the final concentration of sodium bicarbonate and exhibits a better safety.

1. A clear propofol injection comprising the following components: propofol, a cyclodextrin, a stabilizer, a pH regulator and water for injection, and the pH of the injection is 6-9, preferably 7-8.5.

2. The propofol injection according to claim 1, wherein the cyclodextrin is selected from one or more of hydroxypropyl-β-cyclodextrin, sulfobutylether-β-cyclodextrin, hydroxypropyl-sulfobutylether-β-cyclodextrin, mannosyl-β-cyclodextrin and galactosyl-β-cyclodextrin, and preferably hydroxypropyl-β-cyclodextrin or sulfobutylether-β-cyclodextrin.

3. The propofol injection according to claim 1, wherein the stabilizer is selected from pharmaceutically acceptable bases or acids, including but not limited to one or more of sodium bicarbonate, potassium bicarbonate, sodium hydroxide, sodium lactate, sodium hydrogen phosphate, sodium citrate, sodium acetate, ammonium acetate, ammonium bicarbonate, potassium bicarbonate, phosphoric acid, lactic acid, citric acid, acetic acid, hydrochloric acid and carbon dioxide, and preferably sodium bicarbonate;
preferably, the pH regulator is selected from pharmaceutically acceptable bases or acids, including but not limited to sodium bicarbonate, sodium hydroxide, hydrochloric acid and carbon dioxide.

4. The propofol injection according to claim 1, wherein the amount of propofol in the injection is 0.5%-2% (w/v), preferably 1% (w/v) by weight percentage;
preferably, the amount of the cyclodextrin in the injection is 10%-30% (w/v), preferably 12%-25% (w/v) by weight percentage.

5. The propofol injection according to claim 1, wherein the amount of the stabilizer in the injection is 0.001-1.0% (w/v), preferably 0.01-1.0% (w/v), more preferably 0.01-0.5% (w/v) by weight percentage.

6. The propofol injection according to claim 1, wherein the content of free propofol in the injection is not higher than 50 μg/mL, preferably not higher than 35 μg/mL, and most preferably not higher than 30 μg/mL.

7. The propofol injection according to claim 1, wherein the injection further comprises one or more of preservatives, antioxidants, bacteriostatic agents and osmotic adjusters, and the preservative is for example EDTA, the antioxidant is for example sodium bisulfite, and the bacteriostatic agent is for example vitamin C.

8. A clear propofol injection, wherein the injection comprises propofol, sulfobutylether-β-cyclodextrin, sodium bicarbonate, a pH regulator other than sodium bicarbonate and water for injection, and the pH value of the injection is 6-9, preferably 7-8.5;
preferably, the amount of propofol in the injection is 0.5%-2% (w/v), preferably 1% (w/v) by weight percentage;
preferably, the amount of sulfobutylether-β-cyclodextrin in the injection is 10%-30% (w/v), preferably 12%-25% (w/v) by weight percentage.
preferably, the amount of sodium bicarbonate in the injection is 0.001-1.0% (w/v), preferably 0.01-1.0% (w/v), more preferably 0.01-0.5% (w/v) by weight percentage.
preferably, the pH regulator other than sodium bicarbonate is selected from pharmaceutically acceptable bases or acids, including but not limited to sodium hydroxide, hydrochloric acid and carbon dioxide.

9. A preparation method of the propofol injection according to claim 1 comprising the steps of:
adding a prescription amount of cyclodextrin and a stabilizer to water for injection, introducing inert gas after dissolution, adding propofol, stirring, adjusting the pH value with a pH regulator, and filtering;
preferably, the inert gas is nitrogen;
more preferably, the preparation method comprises adding sodium bicarbonate and sulfobutylether-β-cyclodextrin to water for injection, introducing inert gas after dissolution, adding propofol, stirring till the suspended oil droplets of propofol disappear and stirring for another 2-6 hours, adjusting the pH value with a pH regulator, filtering, and blowing nitrogen.

10. A method of anesthesia, the method comprising administering the propofol injection according to claim 1 to a patient in need thereof.

11. A preparation method of the propofol injection according to claim 8 comprising the steps of:
adding a prescription amount of cyclodextrin and a stabilizer to water for injection, introducing inert gas after dissolution, adding propofol, stirring, adjusting the pH value with a pH regulator, and filtering;
preferably, the inert gas is nitrogen;
more preferably, the preparation method comprises adding sodium bicarbonate and sulfobutylether-β-cyclodextrin to water for injection, introducing inert gas after dissolution, adding propofol, stirring till the suspended oil droplets of propofol disappear and stirring for another 2-6 hours, adjusting the pH value with a pH regulator, filtering, and blowing nitrogen.

12. A method of anesthesia, the method comprising administering the propofol injection according to claim 8 to a patient in need thereof.
